(19) United States
(12) Patent Application Publication
(10) Pub. No.: US 2002/0049487 A1
(43) Pub. Date: Apr. 25, 2002

(54) Stress-optimized stent

(75) Inventors: Daniel Lootz, Warnemuende (DE); Curt Kranz, Berlin (DE)

Correspondence Address: HAHN LOESER & PARKS, LLP, TWIN OAKS ESTATE, 1225 W. MARKET STREET, AKRON, OH, 44313, US

(73) Assignee: BIOTRONIK Mess-und Therapiegeraete GmbH & Co Ingenieurbuero Berlin

(21) Appl. No.: 09/939,057

(22) Filed: Aug. 24, 2001

(30) Foreign Application Priority Data
Aug. 30, 2000 (DE) 100 44 043.6

(51) Int. Cl. A61F 2/06

(52) U.S. Cl. 623/1.11; 623/1.16; 623/1.19

(57) ABSTRACT

A stent, in particular a peripheral stent, for expansion from a first condition in which it can be introduced into a vessel (8; 8′″) into a second condition in which it holds the vessel (8; 8′″) expanded, comprising a number of annular support portions (2, 2.1, 2.2) comprising bar elements (3; 3′; 3″; 3′″) which are connected by way of connecting bars (4; 4′) in the longitudinal direction of the stent (1; 1′; 1″; 1′″), wherein the bar elements at least in a portion-wise manner comprise a stent material including a shape memory alloy, characterized in that the width of the bar elements (3; 3′; 3″; 3′″) varies over their length in such a way that the stresses which occur in the bar elements (3; 3′; 3″; 3′″) when the stent material makes the transition from a first structural state into a second structural state, in particular as a result of an increase in temperature, are below the respective plastic deformation limit of the stent material.

The invention concerns a stent, in particular a peripheral stent, for expansion from a first condition in which it can be introduced into a vessel into a second condition in which it holds the vessel in an expanded state, comprising a number of annular support portions comprising bar elements which are connected in the longitudinal direction of the stent by way of connecting bars, wherein the bar elements comprise at least in a portion-wise manner a stent material including a shape memory alloy.

### BACKGROUND OF THE ART

There are two kinds of stents. On the one hand, there are the stents which are frequently referred to as self-expanding and which in their first condition are surrounded by a sheathing device and are elastically compressed thereby, which is then removed from the stent for expansion thereof. On the other hand, there are the stents which are frequently referred to as balloon expansible and which are disposed on an expansible balloon which is expanded for expansion of the stent and in so doing plastically deforms the stent to such a degree that it holds the vessel in the expanded state.

The self-expanding stents are frequently employed for peripheral uses, for example in the region of the carotid arteries or the veins in the legs. In comparison with balloon-expansible stents, they enjoy the advantage that, by virtue of their elastic properties, after unwanted deformation due to external mechanical influences as can certainly occur in respect of peripheral uses, they return of their own accord again to the completely expanded condition in which they hold the vessel in an expanded state.

Self-expanding stents are generally introduced into the vessel in a so-called compression catheter in which they are disposed in a sheathing tube, compressed in a state of elastic deformation to a reduced radius. When the implantation location is reached, the sheathing tube is retracted with respect to the stent and the stent expands of its own accord by virtue of the elastic return forces operative therein.

Such self-expanding stents however suffer from the disadvantage that generally they can only be positioned at the cost of relatively high complication. The correct position thereof in the expanded condition can only be verified with difficulty, before the expansion procedure, that is to say as long as they are in the sheathing tube in their first condition. Once completely expanded, correct positioning of the stent can admittedly be properly checked. However, the stent itself can then only be repositioned with difficulty, if at all. Therefore, correction of the position of the stent is scarcely still possible at that time.

In order to remedy the problem of correct positioning, European patent specification EP 0 657 147 B1, in connection with a vessel implant which is self-expanding in a similar manner, proposes that the expanded implant is retracted into the sheathing tube again upon detection of incorrect positioning by suitable devices in order to re-position the implant.

It has been found in this connection however that, when the implant has to be retracted into the sheathing tube a number of times, the implant experiences permanent plastic deformation effects which impair its expansion capability. That however is to be avoided in any case in the interests of effective engagement.

A comparable problem already arises moreover when the implant is expanded for the first time after it has been positioned for the first time. More specifically, it has been found that the expansion capability upon first expansion is less than that which should actually nominally prevail.

### SUMMARY OF THE INVENTION

Therefore the object of the present invention is to permit simple and reliable positioning of a stent of the general kind set forth, while retaining the expansion capability of the stent.

Based on a stent as set forth in the classifying portion of claim 1, that object is attained by the features recited in the characterizing portion of claim 1.

The invention is based on the technical teaching that particularly simple and reliable positioning of a stent of the general kind set forth is ensured if the width of the bar elements varies over the length thereof in such a way that the stresses which occur in the bar elements when the stent material makes the transition from a first structural state into a second structural state are below the respective plastic deformation limit of the stent material.

It has been found that, by virtue of a suitable variation in the width of the bar elements over their length, it is possible to provide that the bar elements do not experience permanent plastic deformation in the transition from the first to the second structural state. In that respect the variation in the bar width has the advantage that the bar elements of the stent can otherwise retain their configuration which is adapted to the respective properties of the stent.

The transition from the first to the second structural state can result from an increase in temperature. That can be the case for example prior to first expansion of a stent which was introduced into a sheathing tube in a first structural state at a temperature below body temperature and which was then brought to body temperature, for example upon being introduced into the vessel, whereby it then makes the transition into a second structural state. Such a case occurs for example in relation to preferred variants of the stent according to the invention, in which the transition from the first structural state to the second structural state occurs in the first condition of the stent.

Preferably in that respect the first structural state then involves a martensitic state and the second structural state involves a stress-induced martensitic state.

In addition or alternatively however the transition from the first to the second structural state can also result from a change in the stresses prevailing in the stent. That can be the case for example if the stent is retracted once or a number of times, from an at least partially expanded condition, into a sheathing tube or the like, in order to correct its positioning.

In that case the first structural state preferably involves an austenitic state and the second structural state involves a stress-induced martensitic state. That is preferably the case when the stent material at body temperature is in a stress-induced martensitic state in the first condition of the stent and in an austenitic state in the second condition of the stent.

In addition, in preferred variants of the stent according to the invention, the geometry of the bar elements is so selected that the stresses which occur in the bar elements when the stent material, for example as a result of an increase in temperature or as a result of a change in stress, makes the transition from the first structural state into the second structural state, are below the respective plastic deformation limit of the stent material. That affords an additional possible variation.

As mentioned, the invention can be used to particular advantage in connection with a stent which is so designed that, in relation to a sheathing which bears against it at least in a portion-wise manner, the stent is displaceable in a first direction without hooking on the sheathing.

That is particularly advantageous if the stent is already in a condition of being expanded at least in a portion-wise manner and can then be nonetheless displaced in the first direction with respect to the sheathing.

The sheathing may involve a separate sheathing device, such as for example the sheathing catheter of a self-expanding stent, or a corresponding sheathing catheter for a balloon-expansible stent. It can also be formed however by the vessel to be expanded itself, which then bears possibly under a prestressing force against the stent which is expanded at least in a portion-wise manner.

Preferably, the invention is used in conjunction with the variants in which there is provided a sheathing device. Thus for example in the case of self-expanding stents it is provided that, when the sheathing device has not yet been completely removed from the stent, the stent can be restored to its first condition again by producing a relative movement of the sheathing device with respect to the stent in a second direction in opposite relationship to the first direction without hooking on the sheathing device.

The stent can for example simply be withdrawn in the first direction into the sheathing device which is then held in position. That situation does not involve any hooking engagement or comparable effects similarly to the so-called “fish scaling” when introducing conventional balloon-expansible stents without a sheathing catheter.

However the configuration in accordance with the invention is also particularly advantageous when, in relation to a balloon-expansible stent, the first direction is used as the direction of introduction of the stent to the implantation location, as then the above-mentioned effect of “fish scaling”, that is to say hooking on the sheathing which is then formed by the blood vessel, cannot already occur upon introduction of the stent in the non-expanded condition.

Preferably the stent is held in its position and, when dealing with self-expanding stents, the sheathing device is pushed over the stent again or, when dealing with balloon-expansible stents, the sheathing device is possibly even pushed over the stent for the first time, in order in so doing to apply the minimum possible loading to the vessel.

By virtue of the design configuration in accordance with the invention the stent can firstly be expanded in the region of the implantation location and then can be checked in situ by the use of conventional means, in respect of its correct position in relation to the implantation location. In this case, expansion can occur in such a way that the stent has already made the transition into its expanded second condition over a large part of its total length, before the operation for checking correct positioning of the stent is effected. That provides that, in the checking procedure, the stent has already very substantially assumed its actually expanded configuration and therefore, in the checking operation, it is possible to effect more accurate assessment of the later position of the stent.

In order to prevent hooking engagement of the bar elements on the sheathing, for example on the sheathing device, upon being returned to its first condition, the stent can be provided with a casing or enclosure comprising a woven fabric or a foil or sheet. It is then such that on the one hand it makes it possible for the stent to expand. On the other hand it is such that, when the stent reverts, an adequate, radially inwardly directed force component is exerted by way of the enclosure on portions of the bar elements, which project in the first direction, that force component ensuring that, upon reversion of the stent, the stent, in the region immediately adjoining the free end of the sheathing device, is respectively already compressed to such an extent, that is to say, closed down to a reduced diameter, such that it can slide into the sheathing device without hooking engagement or the sheathing device can slide over the stent. The fabric or the foil or sheet in that case only have to be such that in the peripheral direction of the stent they afford sufficient elasticity or are of a sufficient oversize with respect to the stent in its first condition, such as not substantially to hinder expansion of the stent. In contrast, a sufficiently low level of elasticity is required in the longitudinal direction of the stent, in order to ensure the above-mentioned preliminary deformation in the stent region immediately adjoining the free end of the sheathing device, when the stent reverts to the first condition.

Preferably however the bar elements and the connecting bars themselves are already of a suitable configuration and arrangement such as to prevent hooking engagement from occurring. Thus preferably the connecting bars between a first annular support portion and a second annular support portion which is adjacent in the first direction, to prevent hooking engagement between the stent and the sheathing device upon reversion of the stent to its first condition, engage in the region of the portions, which project in the first direction, of the bar elements of the first annular support portion. With these variants, no hooking engagement between the stent and the sheathing device can occur as the connecting bars provide that, when the stent is restored to its first condition, portions of the bar elements, which project in the first direction, in the stent region immediately adjoining the free end of the device, are already drawn radially inwardly to such a degree that they can slide into the sheathing device without hooking or the sheathing device can similarly slide over them.

An embodiment of the stent according to the invention, which is particularly simple to produce, is afforded if at least one first annular support portion and a second annular support portion in adjacent relationship in the first direction are formed by a respective bar element extending in a meander configuration in the peripheral direction of the stent and the connecting bars between the first annular support portion and the second annular support portion engage same in the region of the turning or reversal points, adjoining the second support portion, of the bar element of the first support portion.

Preferably, the respective connecting bar engages the point, which projects furthest in the first direction, of the bar element of the first annular support portion, as that arrangement ensures that in that region there are no portions of the bar element in question, which project in the first direction beyond that force-engagement point which is crucial in respect of the above-mentioned preliminary deformation. This configuration therefore cannot involve any hooking engagement when the stent is restored to its first condition.

In preferred embodiments of the stent according to the invention the connecting bars engage the central region of the second annular support portion in relation to the longitudinal direction of the stent. That ensures that, upon expansion, the stent is reduced in length to the minimum possible degree as a considerable reduction in length upon expansion of the stent is generally unwanted. It will be appreciated however that the connecting bars can also engage any other locations of the second annular support portion, in particular also the end regions thereof, with respect to the longitudinal direction.

Configurations of the stent according to the invention, which are particularly advantageous in this connection because they are simple to produce are distinguished in that at least the second annular support portion is formed by a bar element which extends in a meander configuration in the peripheral direction of the stent and the connecting bars engage in relation to the longitudinal direction of the stent in the central region of the bar element of the second support portion between the turning points of the bar element of the second support portion.

In further advantageous variants of the stent according to the invention the connecting bars are of a sufficient length to ensure flexibility of the stent in relation to its longitudinal direction. That can be guaranteed for example by the respective connecting bar engaging not in the region of the portion of the first bar element which is most closely adjacent in the peripheral direction and which projects in the first direction, but in the region of a correspondingly projecting portion which is displaced in the peripheral direction in relation thereto.

Further advantageous developments of the stent according to the invention are distinguished in that the connecting bars are of such a configuration and arrangement as to avoid twisting of the stent over its length. For that purpose, as considered in the longitudinal direction of the stent, the connecting bars are preferably arranged individually or in a portion-wise manner on alternate sides with respect to a line extending along the longitudinal direction of the stent, in such a way that a change in angle in opposite directions is imparted at least to their engagement points in the first direction on the bar elements upon expansion of the stent, in the tangential plane of the peripheral surface of the stent, individually or in a portion-wise manner. Those oppositely directed changes in angle provide that, upon expansion of the stent, the annular support portions are turned in opposite relationship relative to each in the peripheral direction individually or in a portion-wise manner, with respect to the longitudinal axis of the stent, whereby, as viewed over the entire stent, this preferably affords complete compensation for that turning movement and accordingly therefore twisting of the stent is prevented.

In preferred, in particular self-expanding embodiments of the stent according to the invention the stent material includes a shape memory alloy. This may be for example a copper-based superelastic material. Preferably however a nickel-titanium alloy is used on the grounds of good physiological compatibility. Those shape memory alloys enjoy the advantage that, starting from an original shape, the stent can be plastically deformed at a first temperature and nonetheless returns to its original shape upon an increase in the temperature.

Preferably, as mentioned above, at body temperature the stent material in the first condition of the stent is in a stress-induced martensitic state while in the second condition of the stent it is in an austenitic state. That makes it possible for the stent to be plastically deformed, that is to say compressed, from an initial condition which substantially corresponds to the expanded final condition, at a temperature below body temperature, in such a way that it can be readily introduced into the sheathing device of a suitable catheter at that temperature. An increase in temperature to body temperature provides that the stent thereafter endeavors to return again to its original shape. It is initially prevented from doing so by the sheathing device so that it is in a stress-induced martensitic state. It is only when the sheathing device has been removed that the stent expands, then making the transition into its austenitic state.

Preferred variants of the stent according to the invention, as mentioned above, are distinguished in that the width of the bar elements varies over the length thereof in such a way and possibly in addition the geometry of the bar elements is so selected that the stresses which occur in the bar elements when the stent material which includes a shape memory alloy, in the first condition of the stent, changes from the martensitic state into a stress-induced martensitic state, for example as a result of an increase in temperature, are below the respective plastic deformation limit of the stent material. That ensures that the self-expansible properties of the stent are not adversely affected by plastic deformation of the stent during the transition from the martensitic state into the stress-induced martensitic state. That is advantageous in particular in connection with the stent being returned to its first condition a single time or a plurality of times. Otherwise, it is precisely upon multiple return of the stent to its first condition that progressive plastic deformation effects could be imparted thereto, which ultimately could result in the stent not enjoying complete expansion. The variation in the thickness of the bar elements over the length thereof represents, as mentioned, the underlying concept of the invention here, in relation to shape memory stents. It will further be appreciated that preferably also the width of the connecting bars varies in a corresponding manner over the length thereof.

In variants of the stent according to the invention, which are preferred because they are of a simple structure, at least one annular support portion is formed by a bar element which extends in a meander configuration in the peripheral direction of the stent and the width of which decreases towards the center between two turning points. By virtue of that arrangement, it is easily possible to achieve the above-described limitation in terms of the stresses in the bar element in question as a consequence of the increase in temperature for making the transition to the stress-induced martensitic state.

Further preferred embodiments of the stent according to the invention are distinguished in that at least one annular support portion is formed by a bar element which extends in a meander configuration in the peripheral direction of the stent and the direction of curvature of which changes in the central region between two turning points which are adjacent in the course of the bar element. That arrangement also provides that the stress distribution over the bar element in question is advantageous because it is uniform.

A further influence in terms of stress distribution, which is advantageous in the above-indicated sense, over the bar element in question, is achieved in that at least one annular support portion is formed by a bar element which extends in a meander configuration in the peripheral direction of the stent and in relation to which at least the center line of the bar element is in the shape of a segment of an elliptical arc, in the region of the turning points.

In further variants of the stent according to the invention, which are preferred because they are of a particularly simple structure, at least one annular support portion is formed by a bar element which extends in a meander configuration in the peripheral direction of the stent, wherein each two bar element portions which are adjacent in the peripheral direction of the stent and which extend between the turning points form the limbs of a V.

The invention further concerns a catheter for implanting a stent according to the invention comprising a distal end, in the region of which is provided a sheathing device for receiving the stent in its first condition, and a device for producing the relative movement between the sheathing device and the stent in the first direction. In accordance with the invention that catheter is distinguished in that there are provided a device for producing the relative movement between the sheathing device and the stent in the second direction and a holding device for holding the stent during that relative movement in the second direction. In that way it is easily possible for the stent which is held by the holding device to be restored to its first condition. That can be effected for example by the stent being retracted into the sheathing device, with the sheathing device being held fast, by displacement of the holding device with respect to the sheathing device. It will be appreciated that alternatively the stent can also be held in position by way of the holding device and the sheathing device can be pushed over the stent by means of a suitable device.

Preferably, there are provided a sheathing tube whose distal end forms the sheathing device and a holding element which is arranged displaceable in said sheathing tube for producing the relative movement in the first and second directions, for holding the stent during the relative movement in the second direction. That provides a catheter of a particularly simple configuration.

These catheters can be used both with self-expanding and also balloon-expansible stents.

Preferably a catheter according to the invention is already provided with a stent according to the invention, which is arranged in the sheathing device of the catheter.

The present invention further concerns a method of positioning a stent according to the invention in a vessel. This may involve both positioning the stent in vivo and also in vitro, for example for testing purposes. The method according to the invention provides for example that the self-expanding stent disposed in a sheathing device is moved in a first step in its first condition to the expansion location. Then in a second step the stent is at least partially expanded by at least partial removal of the sheathing device from the stent. In a checking step the position of the stent with respect to the expansion location is detected. In that respect it is provided in accordance with the invention that the stent is only partially expanded in the second step. In at least one correction step the stent is then returned to its first condition again in which it is in the sheathing device and then its position in relation to the expansion location is modified. That correction step can also be repeated a plurality of times before the stent is then definitively completely expanded.

The same method principle can also be implemented with a balloon expansible stent which firstly is moved possibly at least over a part of its length without sheathing device to the implantation location and then repositioned in the above-described manner, using a sheathing device. In that case in the correction step the stent is put into a third condition in which it is arranged in the sheathing device. That third condition can correspond to the first condition. In that respect however, in comparison with its first condition, the stent can also be in a preferably partially expanded condition but also a still further compressed condition.

### BRIEF DESCRIPTION OF THE DRAWINGS

Other advantageous developments of the invention are set forth in the appendant claims or are described in greater detail hereinafter together with the description of the preferred embodiments of the invention, with reference to the accompanying Figures in which:

FIG. 1 shows the development of the peripheral surface of a preferred embodiment of the stent according to the invention,

FIG. 2 is a partly sectional diagrammatic view through an embodiment of a stent according to the invention on a catheter according to the invention in the first condition of the stent,

FIG. 3 shows a view in partial section through the embodiment of FIG. 2 with the sheathing device partially removed,

FIG. 4 shows the development of the peripheral surface of a further preferred embodiment of the stent according to the invention,

FIG. 5 shows the development of a section of a bar element in accordance with a further preferred embodiment of the stent according to the invention, and

FIG. 6 is a view in partial section through a further embodiment of a stent according to the invention on a catheter according to the invention with the sheathing device partially removed.

### DETAILED DESCRIPTION OF THE DRAWINGS

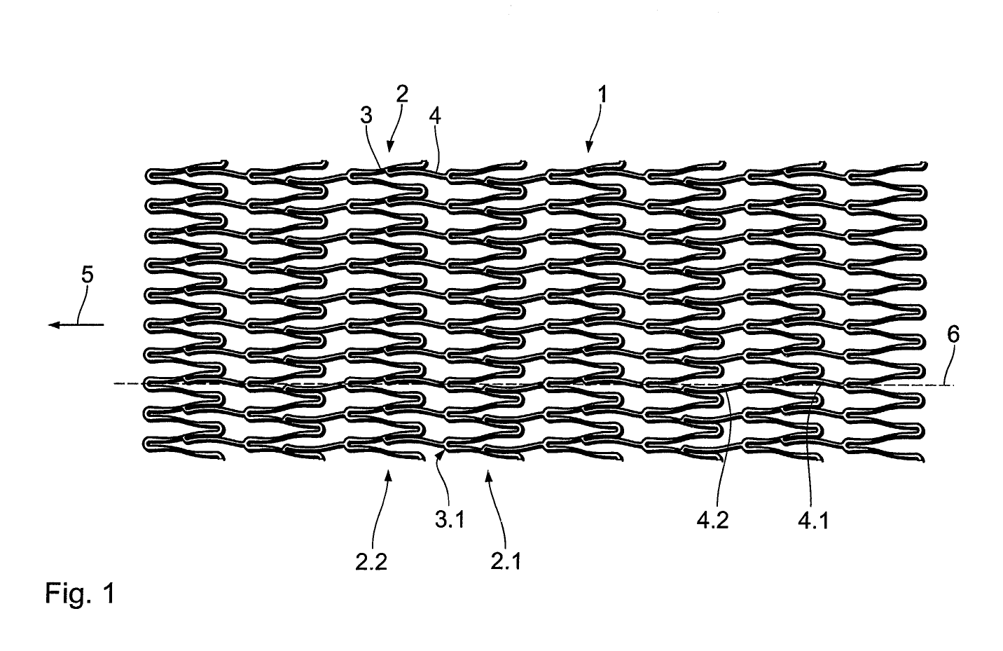

FIG. 1 shows the development of the peripheral surface of a preferred embodiment of the stent 1 according to the invention having a number of annular support portions 2 comprising bar elements 3 which are connected together in the longitudinal direction of the stent 1 by way of connecting bars 4. In other words, the peripheral surface of the stent 1, which in the non-developed condition is formed by a thin-walled tubular component, is of an apertured mesh-like nature. In that arrangement the bar elements 3 and the connecting bars 4 are formed by the remaining wall of the tubular component.

The annular support portions 2 are each formed by a bar element 3 which extends in a meander configuration in the peripheral direction of the stent 1. The connecting bars 4 between a first annular support portion 2.1 and a second annular support portion 2.2 respectively engage the region of the portions, which project in a first direction 5, of the bar elements 3 of the first annular support portion 2.1. In other words, a connecting bar 4 connecting to a bar element which is adjacent in the first direction 5, terminates at each turning point 3.1, which projects in the first direction 5, of a bar element 3 which extends in a meander configuration.

The connecting bars 4 engage the bar elements 3 of the second annular support portion 2.2 in the central region between the turning points of the bar element 3, with respect to the longitudinal direction of the stent. That arrangement ensures that, upon expansion from a first condition in which the stent 1 is compressed to a first diameter into a second condition in which it is expanded to a larger second diameter, the stent 1 is reduced in length only by a minimum amount in its longitudinal direction. That is because, with this configuration, the reduction in length of the respective annular support portion 2 upon expansion of the stent only occurs to such a degree that it is only the change in position of the turning points 3.1 which takes place in the longitudinal direction of the stent 1 with respect to said central region, by way of the connecting bars 4, that contributes to reducing the length of the stent 1. A further effect of this design configuration is that the connecting bars 4 are of a length which ensures flexibility of the stent 1 in relation to its longitudinal direction. The stent 1 can thus adapt well even to severely curved vessels.

In addition, in the longitudinal direction of the stent 1, the connecting bars 4 are arranged on alternate sides with respect to lines 6 extending along the longitudinal direction of the stent 1, in such a way that, upon expansion of the stent 1, a change in angle is imposed on the end point or engagement point, which is in the first direction 5, of a first connecting bar 4.1, which change in angle is in opposite relationship to the change in angle which in that situation is imparted to the end point or engagement point, in the first direction 5, of a second connecting bar 4.2 which is adjacent in the first direction 5. That provides that the annular support portions 2 of the stent 1, upon expansion thereof, admittedly turn relative to each in the peripheral direction of the stent 1, but those rotational movements compensate each other over the length of the stent 1. That ensures that the stent 1 does not experience any twisting effect worth mentioning over its length upon expansion thereof.

In the illustrated example twisting is completely eliminated by virtue of the selected symmetrical configuration and arrangement of the connecting bars 4. It will be appreciated however that in other embodiments of the stent according to the invention, it is also possible to provide other asymmetrical configurations and arrangements of the connecting bars insofar as they afford appropriate compensation for the rotational movements of the annular support portions.

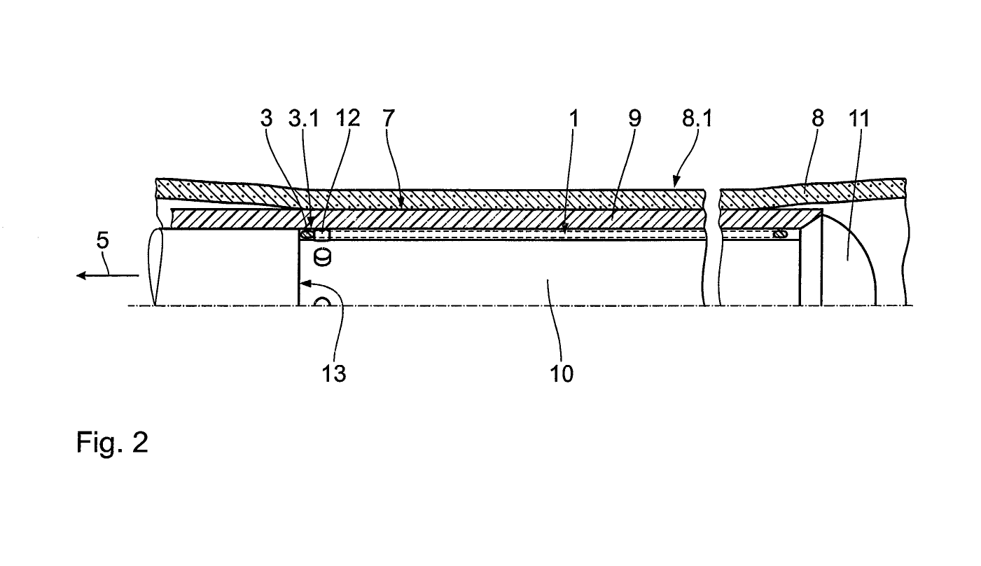

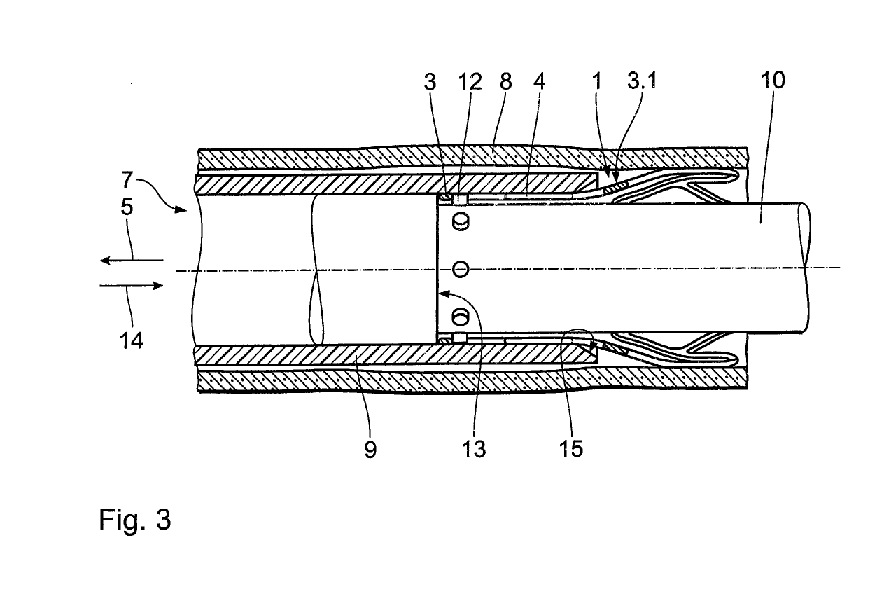

Referring now to FIGS. 2 and 3, the mode of operation of the stent according to the invention and of the catheter according to the invention will be described in greater detail hereinafter.

FIG. 2 diagrammatically shows a partial section of the stent 1 of FIG. 1 on a catheter 7 which has been introduced into a blood vessel 8. In this case the stent 1 is in the region of a constriction 8.1 of the blood vessel 8 which is to be expanded thereby. The stent 1 is shown in FIG. 2 in its first condition in which it is disposed completely in a sheathing device of the catheter 7, which is formed by a sheathing tube 9 arranged at the distal end of the catheter 7. In this case, the stent 1 is in a condition of being compressed to a reduced diameter so that, by virtue of elastic return forces operative therein, it is pressing radially outwardly against the inside wall surface of the sheathing tube 9.

The stent 1 is arranged on a holder 10. The sheathing tube 9 and the holder 10 are arranged displaceable relative to each other. Disposed at the distal end of the holder 10 is a closure cap 11 which in the illustrated condition closes the sheathing tube 9 in order to make it easier to introduce the stent 1 into the blood vessel 8. It will be appreciated however that, in other alternative configurations of the catheter according to the invention, that closure cap does not necessarily have to be provided.

The holder 10 has projections 12 which at the proximal end of the stent 1 engage behind the bar elements 3 in the region of the turning points 3.1 facing in the first direction 5. The holder 10 also has a step 13 serving as an abutment for the stent 1 in the region of the turning points 3.1.

In order to move the stent 1 into its second condition (not shown in FIG. 2) in which it holds the blood vessel 8 in an expanded state the sheathing tube 9 can be withdrawn with respect to the holder 10 and thus also with respect to the stent 1, in the first direction 5. When that happens, the abutment 13 prevents the stent 1 which is biased against the inside wall surface of the sheathing tube 9 being moved with the sheathing tube 9 in the first direction 5. The regions of the stent 1 which are no longer held compressed to the reduced diameter by virtue of removal of the sheathing tube 9 expand immediately. Directly after the sheathing tube 9 has been completely withdrawn from the stent 1 in the first direction 5 the stent 1 is expanded over its entire length and is thus in its second condition.

FIG. 3 is a further partial section through the embodiment of FIG. 2 in a condition of the stent 1 in which the sheathing tube 9 is partially removed from the stent 1, that is to say it has been retracted in the first direction 5 with respect to the stent 1. The portions of the stent 1 which are outside the sheathing tube 9 have already substantially expanded to their final diameter. It is only in the region which directly adjoins the distal end of the sheathing tube 9 that there is a slow transition from the compressed diameter to the expanded diameter, in the longitudinal direction of the stent 1.

As can be seen from FIG. 3 only a small part of the stent 1 is still in the sheathing tube 9. Consequently a large part of the stent 1 is already completely expanded. In that condition, when checking the positioning of the stent 1 in relation to the constriction 8.1 in the blood vessel 8, a very substantially unfalsified picture of the later position of the completely expanded stent 1 is obtained, as, upon further expansion of the small remaining part of the stent 1 which has still remained in the sheathing tube 9, there is no longer any substantial modification in the position of the stent.

In accordance with the method according to the invention it is now possible to effect a correction step in which the partially expanded stent is restored again to its first condition and then its position is corrected. It will be appreciated in that respect that this method does not necessarily have to be executed in vivo, that is to say on the patient. It can also be executed in vitro, that is to say on any other vessels or the like.

Restoration of the stent 1 to its first condition is effected by the sheathing tube 9 being pushed over the stent 1 again relative to the holder 10, in a second direction 14 opposite to the first direction, by means of a device (not shown in FIGS. 2 and 3) at the proximal end of the catheter 7. The projections 12 which engage behind the bar elements 3 at the proximal end of the stent 1 hold the stent 1 in its position with respect to the holder 10 and thereby ensure that the stent is returned to its compressed condition by the leading distal end of the sheathing tube 9, as is shown in FIG. 2.

In that respect the configuration and arrangement of the bar elements 3 and the connecting bars 4 ensure that no hooking engagement can occur in respect of the portions which project in the first direction 5, that is to say the turning points 3.1, projecting in the first direction 5, of the bar elements 3 at the leading distal end of the sheathing tube 9. In that situation the connecting bars 4 ensure that the turning points 3.1 of the bar elements 3, which project in the first direction, when the sheathing tube 9 is pushed over the stent 1, are already drawn radially inwardly in the region of the stent 1 which directly adjoins the leading end of the sheathing tube 9, to such a degree that the sheathing tube 9 can slide without hooking engagement over the portions of the bar elements 3, which project in the first direction 5. In that situation, friction-free movement of the sheathing tube 9 as it is pushed over the stent 1 is further promoted by a bevel 15 at the distal end of the sheathing tube 9.

In the illustrated embodiment the connecting bars 4 engage directly in the region of the turning points 3.1 of the bar elements 3, which face in the first direction 5. In other words, the connecting bars 4 directly engage the portion of the respective bar element 3, which projects furthest in the first direction 5. It will be appreciated however that, in other configurations of the stent according to the invention, the connecting bars 4 do not necessarily have to engage that portion of the respective bar element, which projects furthest in the first direction. In the region thereof they may also engage a portion of the respective bar element, which projects less far in the first direction. In other words, the point of engagement of the respective connecting bar can still be surpassed in the first direction by adjoining portions of the bar element. It is only necessary to ensure that those portions of the bar element, which surpass the point of engagement in the first direction, are drawn radially inwardly by way of the connecting bars when the stent is restored to its first condition, to such an extent that the sheathing device, for example therefore the sheathing tube, can slide over those portions without involving hooking engagement.

It will further be appreciated that the specified principle that portions of the bar elements which project in the first direction are to be drawn radially inwardly by way of the respective connecting bars to such an extent that the sheathing device can slide over those portions without involving hooking engagement is not limited to the meander-shaped bar elements shown in FIG. 1, but can also be applied to any bar elements of a different configuration.

It will further be appreciated that the above-described principle of the method can also be carried into effect with a balloon-expansible stent which, carried on a suitable balloon, in the sheathing device, is moved to the implantation location, then partially expanded by the balloon, that is to say put into the condition shown in FIG. 3, and repositioned using the sheathing device in the manner described hereinbefore.

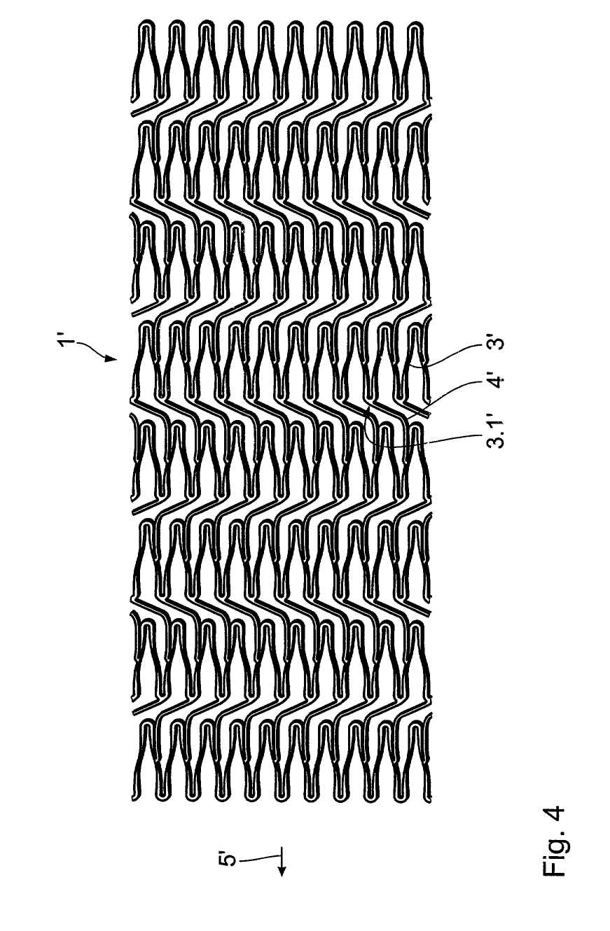

FIG. 4 shows the development of the peripheral surface of another embodiment of the stent according to the invention. In terms of its basic structure this is the same as that shown in FIG. 1 so that only the differences will be discussed herein.

The difference is that the connecting bars 4′ do not directly engage the turning point 3.1′ of the bar element 3′, which is most closely adjacent in the longitudinal direction of the stent 1′, but rather they engage a turning point 3.1′ which is displaced in relation to thereto in the peripheral direction of the stent 1′. In that way the length of the connecting bars 4′ is increased, in comparison with the structure shown in FIG. 1, which in turn results in an increase in the flexibility of the stent 1′ in relation to its longitudinal direction.

The stents shown in FIGS. 1 through 4 each comprise a shape memory alloy on a nickel-titanium basis, referred to as Nitinol. That stent material is in a stress-induced martensitic state at body temperature in the first condition of the stent 1, that is to say in its condition of being compressed in the sheathing tube 9. It is in an austenitic state in the second condition of the stent, that is to say when the stent 1 is substantially relieved of stress. Upon manufacture or prior to its use the stent 1 is plastically deformed, that is to say compressed, from an initial condition which substantially corresponds to the expanded final condition, at a temperature which is below body temperature and at which it is in a martensitic state, in such a way that it can be readily introduced into the sheathing tube 9 of the catheter 7 at that temperature. An increase in the temperature to body temperature then provides that the stent thereafter endeavors to return to its original shape again. It is firstly prevented from doing that by the sheathing tube 9 so that it is in a stress-induced martensitic state. It is only when the sheathing tube 9 is removed that the stent 1 expands and thus passes into its austenitic condition.

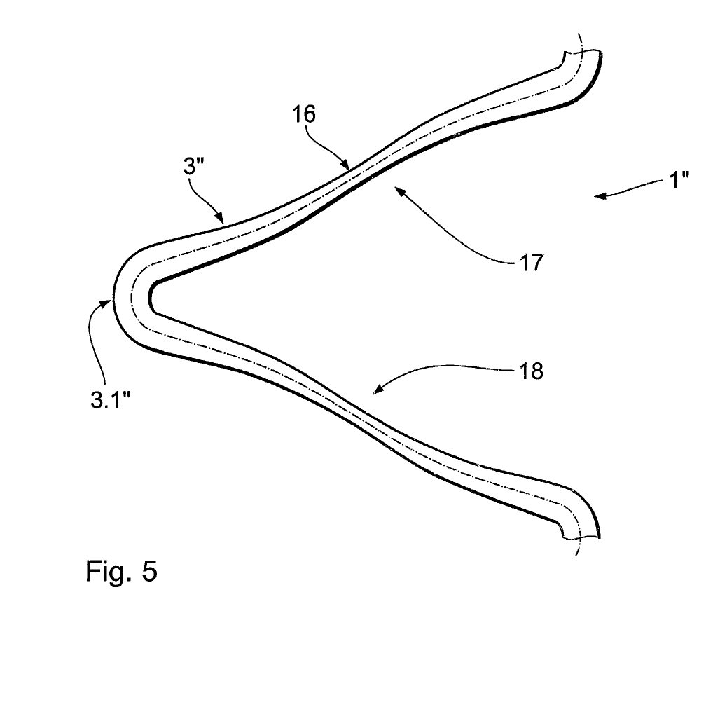

The configuration in respect of thickness and the geometry of the bar elements in FIGS. 1 through 4 correspond to those which are described hereinafter with reference to FIG. 5.

FIG. 5 shows the development of a section of a bar element 3″ in accordance with a preferred embodiment of the stent according to the invention. This also involves a structure comprising one of the above-described shape memory alloys. The stent of FIG. 5 can substantially correspond to the stents shown in FIGS. 1 through 4 so that only the particular features of the bar elements will be discussed here.

The particularity of the bar element 3″ is that on the one hand its geometry is so selected and on the other hand the width of the bar element 3″ varies over its length in such a way that the stresses which occur therein when the stent material in the first condition of the stent 1″ makes the transition from the martensitic state into a stress-induced martensitic state as a result of an increase in temperature remain below the plastic deformation limit of the stent material, which prevails at the respective temperature involved.

That advantageous stress distribution is achieved on the one hand by virtue of the fact that the width of the bar element 3″ respectively continuously decreases towards the center 16 between two turning points 3.1″. In the illustrated example the reduction in thickness is about 50%. In other design configurations of the bar element however it is determined, in dependence on the rest of the geometry of the bar element, in accordance with the respective upper stress limit to be observed.

A further advantageous influence on stress distribution within the bar element 3″ is afforded by virtue of the fact that the direction of curvature of the bar element 3″ changes in the central region 16 between two turning points 3.1″. Therefore, each two bar element portions 17 and 18 which are adjacent in the peripheral direction of the stent 1″ and which extend between the turning points 3.1″ form the curved limbs of a V-shape.

An additional influence on stress distribution over the bar element 3″, which is advantageous along the above-indicated lines, is afforded by virtue of the fact that, in the region of the turning points 3.1″, the bar element 3″ is in the shape of a segment of an elliptical arc, in place of the usual segment of a circular arc.

It will be appreciated that the underlying principle of so selecting the widthwise configuration and possibly additionally the geometry of the bar elements, that the stresses in the bar element remain below the respective plastic deformation limit on a transition being made from the martensitic state into a stress-induced martensitic state can also be applied to any other configurations of the bar elements, irrespective of the basic geometries of the bar elements, as described with reference to FIGS. 1 through 5.

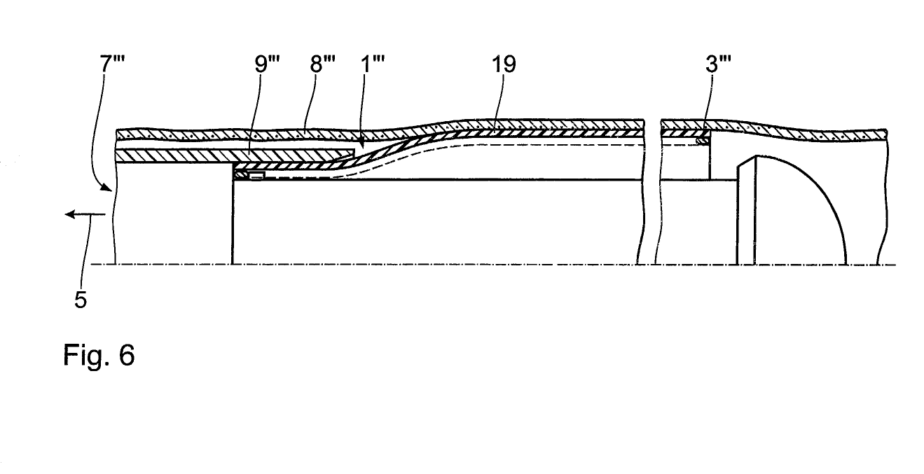

FIG. 6 shows a partial view in section through a further embodiment of a stent according to the invention on a catheter 7′″ according to the invention in the partially expanded condition. In this case the catheter 7′″ substantially corresponds to the catheter shown in FIGS. 2 and 3 so that only the differences in relation to the stent 1′″ will be discussed here.

The difference is that the stent 1′″ is provided with a casing or enclosure 19 to prevent hooking engagement of the bar elements 3′″ on the sheathing tube 9′″ when it is restored to its first condition. The enclosure 19 is so designed that on the one hand it permits expansion of the stent 1′″ into the desired final condition thereof. On the other hand, it is of such a design configuration that, upon the stent 1′″ being restored to its first condition, an adequate radially inwardly directed force component is applied by way of the enclosure to the portions of the bar elements 3′″, projecting in the first direction 5′″, which force component ensures that, when the stent 1′″ is restored to its first condition, the stent 1′″ is respectively already compressed in the region directly adjoining the free distal end of the sheathing tube 9′″, that is to say, it is set to a reduced diameter, such that the sheathing tube 9′″ can slide over the stent 1′″ without hooking engagement occurring.

For that purpose, in the peripheral direction of the stent the enclosure 19 has adequate elasticity which substantially does not impede expansion of the stent. In contrast, in the longitudinal direction of the stent it is of low elasticity in order to ensure the above-mentioned preliminary deformation in the region of the stent 1′″ directly adjoining the free end of the sheathing tube, when the stent is restored to the first condition thereof. In the illustrated example that is achieved by means of a sheet or foil 19 of suitably elastic plastic material in which are embedded fibers which are of suitable tensile strength and which extend in the longitudinal direction of the stent. The tensile fibers extend in the region of the portions of the bar elements 3′″, which project in the first direction 5′″. In that region the bar elements 3′″ are also connected to the foil or sheet 19 in order to ensure a uniform application of force or to prevent the fibers sliding away from those regions where the force is applied.

The second material can comprise one or more components which is or are identical to none of the components of the first material.

The second material however may equally well correspond to a component of the first material. When the first material is made up from a matrix in which the first substance is incorporated, the second material may for example consist of or correspond to the component of the first material, which forms the matrix.

1. A stent, in particular a peripheral stent, for expansion from a first condition in which it can be introduced into a vessel (8; 8′″) into a second condition in which it holds the vessel (8; 8′″) in an expanded state, comprising a number of annular support portions (2, 2.1, 2.2) comprising bar elements (3; 3′; 3″; 3′″) which are connected in the longitudinal direction of the stent (1; 1′; 1′″ 1′″) by way of connecting bars (4; 4′), wherein the bar elements comprise at least in a portion-wise manner a stent material including a shape memory alloy, characterized in that the width of the bar elements (3; 3′; 3″; 3′″) varies over their length in such a way that the stresses which occur in the bar elements (3; 3′; 3″; 3′″) when the stent material makes the transition from a first structural state into a second structural state, in particular as a result of an increase in temperature, are below the respective deformation limit of the stent material.

2. A stent as set forth in claim 1 characterized in that the first structural state is a martensitic state and the second structural state is a stress-induced martensitic state.

3. A stent as set forth in claim 1 or claim 2 characterized in that the transition from the first structural state into the second structural state occurs in the first condition of the stent (1; 1′; 1″; 1′″).

4. A stent as set forth in one of the preceding claims characterized in that the stent material includes a nickel-titanium alloy.

5. A stent as set forth in one of the preceding claims characterized in that at body temperature the stent material is in a stress-induced martensitic state in the first condition of the stent (1; 1′; 1″; 1′″) and in an austenitic state in the second condition of the stent (1; 1′; 1″; 1′″).

6. A stent as set forth in one of the preceding claims characterized in that the geometry of the bar elements (3; 3′; 3″; 3′″) is so selected that the stresses which occur in the bar elements (3; 3′; 3″; 3′″) when the stent material makes the transition from the first structural state into the second structural state, in particular as a result of an increase in temperature, are below the respective plastic deformation limit of the stent material.

7. A stent as set forth in one of the preceding claims characterized in that at least one annular support portion is formed by a bar element which extends in a meander configuration in the peripheral direction of the stent (1; 1′; 1″; 1′″) and whose width decreases towards the center (16) between two turning points (3.1; 3.1′; 3.1″).

8. A stent as set forth in one of the preceding claims characterized in that at least one annular support portion (2, 2.1, 2.2) is formed by a bar element (3; 3′; 3″; 3′″) which extends in a meander configuration in the peripheral direction of the stent (1; 1′; 1″; 1′″) and whose direction of curvature changes in the central region (16) between two turning points (3.1; 3.1′; 3.1″).

9. A stent as set forth in one of the preceding claims characterized in that at least one annular support portion (2, 2.1, 2.2) is formed by a bar element (3; 3′; 3″; 3′″) which extends in a meander configuration in the peripheral direction of the stent (1; 1′; 1″; 1′″) and of which at least the center line is in the shape of a segment of an elliptical arc in the region of the turning points (3.1; 3.1′; 3.1″).

10. A stent as set forth in one of the preceding claims characterized in that at least one annular support portion (2, 2.1, 2.2) is formed by a bar element (3; 3′; 3″; 3′″) which extends in a meander configuration in the peripheral direction of the stent (1; 1′; 1″; 1′″) wherein each two bar element portions (17, 18) which are adjacent in the peripheral direction of the stent (1; 1′; 1″; 1′″) and which extend between the turning points (3.1; 3.1′; 3.1″) form the limbs of a V-shape.

11. A stent as set forth in one of the preceding claims characterized in that the bar elements (3; 3′; 3′″) and the connecting bars (4; 4′) are of such a configuration and arrangement that the stent (1; 1′; 1″) is displaceable with respect to a sheathing (8, 9; 8′″; 9′″) bearing at least in a portion-wise manner thereagainst, in particular in a condition of being expanded at least in a portion-wise manner, in a first direction (5; 5′; 5′″ 5′″), without hooking engagement on the sheathing (8, 9; 8′″; 9′″).

12. A stent as set forth in claim 11 characterized in that the connecting bars (4; 4′) between a first annular support portion (2.1) and a second annular support portion (2.2) which is in adjacent relationship in the direction of displacement (5; 5′) engage in the region of the portions, projecting in the first direction (5; 5′) of the bar elements (3; 3′) of the first annular support portion (2.1), for preventing hooking engagement between the stent (1; 1′) and the sheathing (8, 9) upon displacement of the stent (1; 1′).

13. A stent as set forth in claim 11 or claim 12 characterized in that at least a first annular support portion (2.1) and a second annular support portion (2.2) in adjacent relationship in the first direction (5; 5′) are each formed by a respective bar element (3; 3′) extending in a meander configuration in the peripheral direction of the stent (1; 1′) and the connecting bars (4; 4′) between the first annular support portion (2.1) and the second annular support portion (2.2) engage in the region of the turning points, adjoining the second support portion (2.2), of the bar element (3; 3′) of the first support portion (2.1).

14. A stent as set forth in one of claims 12 and 13 characterized in that the connecting bars (4; 4′) engage the central region of the second annular support portion (2.2) with respect to the longitudinal direction of the stent (1; 1′), in particular the central region of the bar element (3; 3′) of the second annular support portion (2.2) between the turning points (3.1; 3.1′) of the bar element (3; 3′).

15. A stent as set forth in one of the preceding claims characterized in that the connecting bars (4; 4′) are of a sufficient length which ensures flexibility of the stent (1; 1′) with respect to its longitudinal direction and/or are designed and arranged to avoid twisting of the stent (1; 1′) over its length.

16. A catheter arrangement comprising a catheter (7; 7′″) and a stent as set forth in one of the preceding claims, wherein the catheter has a distal end, in the region of which there is provided a sheathing device (9; 9′″) for receiving the stent (1; 1′; 1″; 1′″) in its first condition, and a device for producing the relative movement between the sheathing device (9; 9′″) and the stent (1; 1′; 1″; 1′″) in a first direction (5; 5′; 5″; 5′″).
